(19) United States
(12) Patent Application Publication
(10) Pub. No.: US 2020/0205550 A1
(43) Pub. Date: Jul. 2, 2020

(54) BEAD CREAM COSMETIC STORAGE CONTAINER

(71) Applicant: TAESUNG INDUSTRIAL CO., LTD., Anyang-si, Gyeonggi-do (KR)

(72) Inventor: Kam Sub SONG, Yongin-si, Gyeonggi-do (KR)

(21) Appl. No.: 16/623,452

(22) PCT Filed: May 10, 2018
(86) PCT No.: PCT/KR2018/005354

§ 371 Date: Dec. 17, 2019

(30) Foreign Application Priority Data
Jun. 30, 2017 (KR) 10-2017-0083406

(51) Int. Cl. A45D 40/00; A61K 8/92; A61K 8/68; A61Q 19/08; A61Q 19/00; A61K 8/02

(52) U.S. Cl. CPC A45D 40/0075; A61K 8/92; A61K 8/0241; A61Q 19/08; A61Q 19/007; A61K 8/68

(57) ABSTRACT

Provided is a bead cream cosmetic storage container enabling bead cream cosmetics to be taken out and used without breakage when the bead cream cosmetics, which are formed by enclosing, with wax, ceramides having the effects of effectively inhibiting evaporation of moisture in the skin and, simultaneously repairing the damaged skin bather quickly, are taken out and used. The container is formed such that the bead cream cosmetics accommodated in a storage space can be used by being individually dispensed to a discharge recessed portion formed on the upper surface of an intermediate cap by using a dispensing rod which is positioned in a dispensing rod lifting and lowering cylinder of a rotating body to penetrate a dispensing rod guide tube formed in the center of the bottom surface of a main body and having a cone shape, and is lifted and lowered by a rotational motion of the rotating body.

### TECHNICAL FIELD

The present invention relates generally to a container for storing cosmetic creams used to suppress evaporation of moisture from skin and quickly restore a damaged skin barrier to reduce facial wrinkles and neck wrinkles. More particularly, the present invention relates to a bead cream cosmetic storage container enabling cosmetic cream beads to be taken out for use without breakage of the beads when the cosmetic cream beads, which are formed by enclosing, with wax, ceramides having effects of effectively suppressing evaporation of moisture from skin and, simultaneously repairing a damaged skin barrier quickly, are taken out for use.

### BACKGROUND ART

Modern people spend a lot of time and effort caring for themselves regardless of age or gender.

In particular, efforts to suppress skin aging caused by evaporation of moisture in the skin have been tried in various ways, one of which is the use of functional cosmetics.

Human skin consists of three layers: the epidermis, the dermis, and the subcutaneous tissue. The stratum corneum, which is the outermost layer of the epidermis, performs a protective function of skin. Lipids, which account for 15% of the stratum corneum, consist of ceramides, fatty acids, and cholesterol.

Of these, ceramides consists of approximately 50% of the lipids, and ceramides has a function of suppressing evaporation of moisture and maintaining an orderly structure of the stratum corneum. In the stratum corneum, keratinocytes are firmly bound to each other by ceramides, cholesterol, and free fatty acids. Skin cells have a higher ceramide content than other cells of human body and have a high content of non-polar neutral ceramides. This is a structural feature that is necessary to construct a solid protective barrier of skin.

Ceramide can be decomposed on the surface of skin to generate phytosphingosine, acting as an antimicrobial barrier against external harmful microorganisms, and also plays a vital role in controlling inflammation and healing wounds.

Skin contains both sebum and moisture, and an appropriate amount of the oil component functions to suppress evaporation of moisture through skin.

A lipid layer on the epidermis of skin, along with sebum, also plays a role in preventing loss of moisture in the skin. A major component of this lipid layer is ceramide.

Ceramide has both a hydrophilic group and a lipophilic group in the chemical structure and thus functions to prevent moisture in skin from evaporating.

When skin lacks ceramide, which is the main component of intercellular lipids, the formation of intercellular lipids may be inhibited. A lack of intercellular lipids may lead to skin dryness, dry skin, skin eczema, rough skin, atopic skin, accelerated skin aging, and the like.

Existing creams and lotions currently on the market have very limited ceramide contents due to poor solubility of ceramide, which is not helpful for reducing facial wrinkles and neck wrinkles by suppressing evaporation of moisture from skin and quickly restoring damaged skin barriers.

In an effort to solve such a drawback of the existing creams and lotions, a bead cream (hereinafter referred to as “cosmetic cream bead”) has been developed.

As shown in FIG. 1, a cosmetic cream bead 10 includes an outer wax layer 12 and a high content of ceramide 11 contained therein in emulsion form.

Cosmetic cream beads 10 are individually taken out for use. A container 1 in which the cosmetic cream beads are stored is the same as a general cream-type cosmetic container as shown in FIG. 2. Due to this, taking out of each cosmetic cream bead requires the use of a tool 2 such as tweezers as shown in FIG. 3.

This is because the cosmetic cream beads are formed by enclosing ceramides with soft wax, so when a user takes out each cosmetic cream bead by a hand, other cosmetic cream beads surrounding the bead to be taken out may contact with his or her fingers and be crushed thereby, leading to breakage.

Although such a problem can be solved by the use of tweezers, a cumbersome manufacturing process is required, such as having to separately provide a space in the container for provision of a tool (tweezers) for taking out the cosmetic cream beads. In addition, careless storage during use may cause the tool for taking out the cosmetic cream beads to become contaminated with foreign substances, causing skin problems that are incompatible with skin care.

### DISCLOSURE

### Technical Problem

The present invention has been developed in order to solve the problems occurring in the related art such as breakage of cosmetic cream beads occurring when taking out the cosmetic cream beads from a container, and an objective of the present invention is to provide a bead cream cosmetic storage container configured such that cosmetic cream beads accommodated in a storage space are used by being individually dispensed to a discharge recessed portion formed on an upper surface of an intermediate cap by using a dispensing rod which is positioned in a dispensing rod lifting cylinder of a rotatable body by passing a dispensing rod guide tube formed in the center of a conical-shaped bottom surface of a main body, and is lifted or lowered in conjunction with a rotational motion of the rotatable body. Therefore, the present invention not only fundamentally solves the problems of the cosmetic container in the related art in which when a cosmetic cream bead is taken out for use from the container, other cosmetic cream beads surrounding the bead to be taken out may be broken, but also eliminating the use of a tool to thereby eliminate a cumbersome manufacturing process of providing a space for storing the tool in the container. In addition, the present invention fundamentally solves the problems that the cosmetic cream beads may be contaminated with contaminants when the cosmetic cream beads are taken out with a tool contaminated due to careless storage, and that skin troubles may be caused by the use of the contaminated cosmetic cream beads.

### Technical Solution

In order to accomplish the above objective, the present invention provides a bead cream cosmetic storage container that stores and uses, among cosmetic creams used to suppress evaporation of moisture from skin and quickly restore a damaged skin barrier to reduce facial wrinkles and neck wrinkles, cosmetic cream beads each of which includes an outer wax layer and a high content of ceramide contained therein in emulsion form, the bead cream cosmetic storage container including: a main body configured such that a bottom surface forming a space where the cosmetic cream beads are stored has a conical shape to allow the cosmetic cream beads to be gathered to a center of the bottom surface, a dispensing rod guide tube in which a dispensing rod pushing up the cosmetic cream beads is positioned to be lifted or lowered is provided in the center of the bottom surface, a rotatable body configured to lift or lower the dispensing rod is assembled to the main body so as to be rotatable via a lower coupling portion, and an intermediate cap is coupled to an upper coupling portion, the intermediate cap including an upper surface provided with a discharge recessed portion through which the cosmetic cream beads dispensed by the dispensing rod are discharged;

the rotatable body configured such that an upper end thereof is coupled to the dispensing rod guide tube of the main body by concave-convex engagement while an inside center thereof is provided with a dispensing rod lifting cylinder including a lifting screw hole that is defined by an inner surface thereof and along which a guide protrusion of the dispensing rod is guided, and assembled to the main body by locking engagement of an annular locking portion formed on an inner surface of an edge wall with an annular locking portion formed on an outer surface of the lower coupling portion of the main body;

the dispensing rod configured such that guide protrusions guided along the lifting screw hole of the dispensing rod lifting cylinder of the rotatable body are formed on a lower outer surface of the dispensing rod at positions opposed to each other, and a bead seat recess in which each of the cosmetic cream beads is positioned to be individually dispensed is formed in an upper end surface of the dispensing rod; and

the intermediate cap configured such that a protective cap is fastened to the intermediate cap by screw engagement so as to protect the intermediate cap against dust and the like, an annular coupling rim is fastened to the upper coupling portion of the main body by undercut engagement, and the discharge recessed portion through which the cosmetic cream beads dispensed by the dispensing rod are exposed is formed on the upper surface of the intermediate cap.

The dispensing rod guide tube of the main body in which the dispensing rod is lifted or lowered may have an inner diameter corresponding to an outer diameter of the dispensing rod, with a D-cut surface corresponding to a D-cut surface formed on an outer circumferential surface of the dispensing rod such that the dispensing rod can be lifted or lowered without rotation.

The bead cream cosmetic storage container may further include a hermetic sealing ring interposed between an upper end of the dispensing rod lifting cylinder of the rotatable body and the dispensing rod guide tube provided in the center of the bottom surface of the main body.

### Advantageous Effects

As described above, a bead cream cosmetic storage container according to the present invention is configured such that cosmetic cream beads accommodated in a storage space are used by being individually dispensed to a discharge recessed portion 132 formed on an upper surface 131 of an intermediate cap 130 by using a dispensing rod 140 which is positioned in a dispensing rod lifting cylinder 121 of a rotatable body 120 by passing a dispensing rod guide tube 112 formed in the center of a conical-shaped bottom surface 111 of a main body 110, and is lifted or lowered in conjunction with a rotational motion of the rotatable body 120, thus realizing the following effects.

First, the present invention not only can fundamentally solve the problems of a cosmetic container in the related art in which when a cosmetic cream bead 10 is taken out for use from the container, other cosmetic cream beads surrounding the bead to be taken out may be broken.

Second, the present invention can eliminate the use of a tool 2 to thereby eliminate a cumbersome manufacturing process of providing a space for storing the tool 2 in the container.

Third, the present invention can fundamentally solve the problems that the cosmetic cream beads may be contaminated with contaminants when the cosmetic cream beads 10 are taken out with a tool 2 contaminated due to careless storage, and that skin troubles may be caused by the use of the contaminated cosmetic cream beads.

### DESCRIPTION OF DRAWINGS

FIG. 1 is a view showing an embodiment of a cosmetic container in the related art in which cosmetic cream beads are stored, and FIG. 2 is a sectional view showing configuration of a cosmetic cream bead.

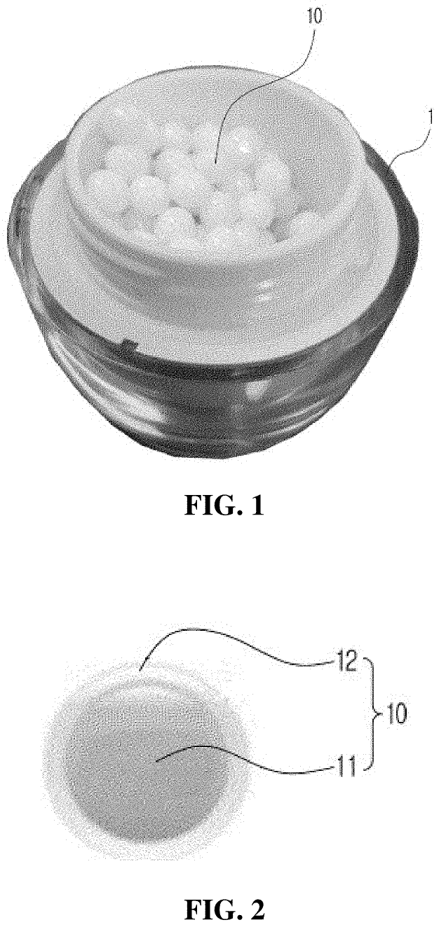

FIG. 3 is an embodiment showing a state in which the cosmetic cream bead is taken out using a tool.

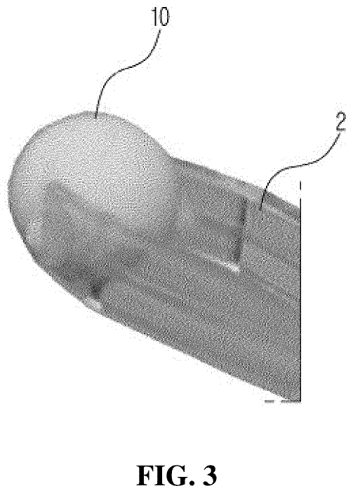

FIG. 4 is a view showing configuration of a bead cream cosmetic storage container according to the present invention.

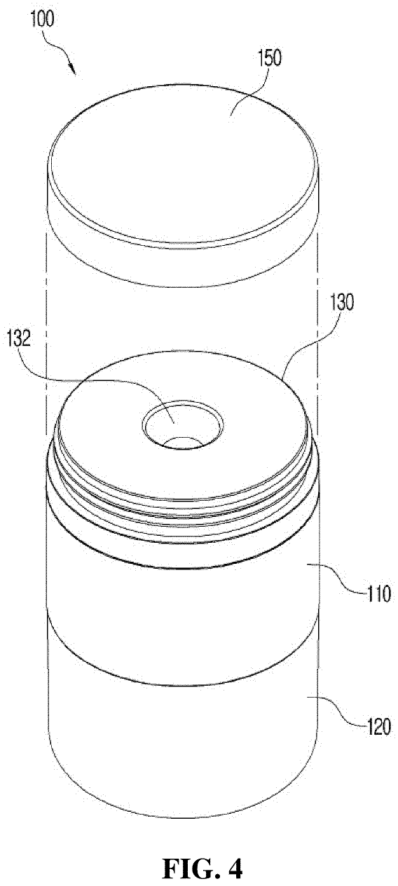

FIG. 5 an exploded sectional view showing configuration of the bead cream cosmetic storage container according to the present invention.

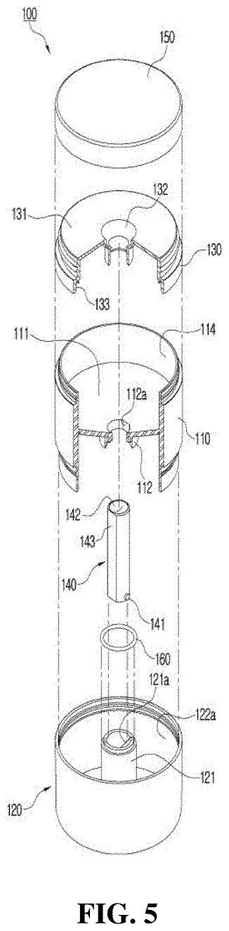

FIG. 6 an exploded sectional view showing configuration of the bead cream cosmetic storage container according to the present invention.

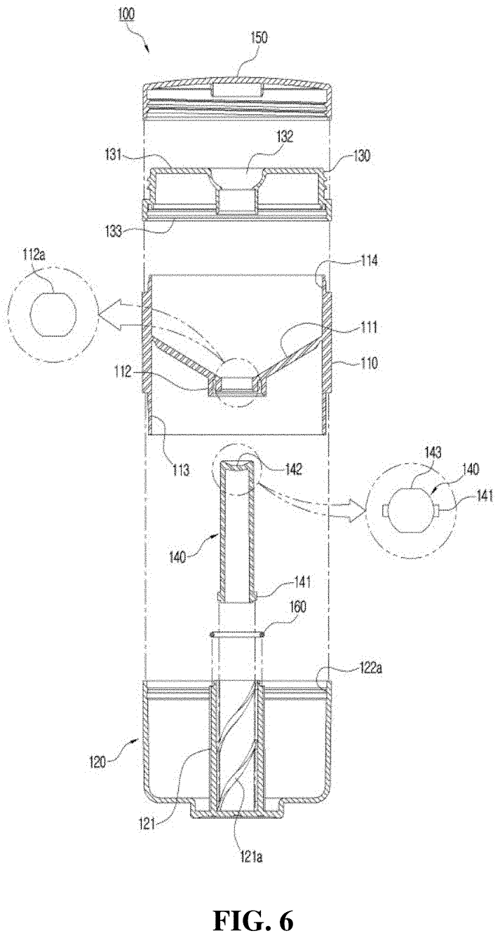

FIG. 7 is an exemplary sectional view showing a state in which cosmetic cream beads are stored in the bead cream cosmetic storage container according to the present invention.

FIG. 8 is an exemplary view showing a state in which each of the cosmetic cream beads stored in the bead cream cosmetic storage container according to the present invention is taken out for use.

### DESCRIPTION OF THE REFERENCE NUMERALS IN THE DRAWINGS

10: cosmetic cream bead 100: bead cream cosmetic storage container
110: main body 111: bottom surface
112: dispensing rod guide tube 112a,143: D-cut surface
113,114: coupling portion 120: rotatable body
121: dispensing rod lifting cylinder 121a: lifting screw hole
122: edge wall 130: intermediate cap
131: upper surface 132: discharge recessed portion
133: annular coupling rim 140: dispensing rod
141: guide protrusion 142: bead seat recess
150: protective cap 160: hermetic sealing ring

### BEST MODE

Hereinafter, an exemplary embodiment for achieving the above objective will be described in detail with reference to the accompanying drawings.

A bead cream cosmetic storage container 100 according to the present invention stores and uses, among cosmetic creams used to suppress evaporation of moisture from skin and quickly restore a damaged skin barrier to reduce facial wrinkles and neck wrinkles, cosmetic cream beads 10 each of which includes an outer wax layer 12 and a high content of ceramide 11 contained therein in emulsion form.

The bead cream cosmetic storage container includes a main body 110 in which the cosmetic cream beads 10 are accommodated, a rotatable body 120 assembled to a lower portion of the main body 10 and configured to lift or lower a dispensing rod 140 configured to individually dispense the cosmetic cream beads 10, and an intermediate cap 130 fastened to an upper portion of the main body 110 and in which the cosmetic cream beads 10 selectively dispensed by the dispensing rod 140 are positioned to be discharged therethrough.

The main body 110 is configured such that a bottom surface 111 forming a space where the cosmetic cream beads 10 are stored has a conical shape and the cosmetic cream beads 10 are gathered to the center of the bottom surface 111.

A dispensing rod guide tube 112 in which the dispensing rod 140 pushing up the cosmetic cream beads 10 is positioned to be lifted or lowered is provided in the center of the bottom surface 111.

The rotatable body 120 configured to lift or lower the dispensing rod 140 is assembled to the lower portion of the main body 110 so as to be rotatable via a lower coupling portion 113.

The intermediate cap 130 is coupled to an upper coupling portion 114, the intermediate cap including an upper surface 131 provided with a discharge recessed portion 132 through which the cosmetic cream beads 10 dispensed by the dispensing rod 140 are discharged.

The rotatable body 120 fastened to the lower portion of the main body 110 is configured such that an upper end thereof is coupled to the dispensing rod guide tube 112 of the main body 110 by concave-convex engagement while an inside center thereof is provided with a dispensing rod lifting cylinder 121 including a lifting screw hole 121a that is defined by the inner surface thereof and along which a guide protrusion 141 of the dispensing rod 140 is guided.

The rotatable body 120 is assembled to the main body by locking engagement of an annular locking portion 122a formed on the inner surface of an edge wall 122 with an annular locking portion (not designated by a specific reference numeral in the drawing) formed on the outer surface of the lower coupling portion 113 of the main body 110.

The dispensing rod 140 positioned in the dispensing rod lifting cylinder 121 of the rotatable body 120 is configured such that guide protrusions 141 guided along the lifting screw hole 121a of the dispensing rod lifting cylinder 121 of the rotatable body 120 are formed on a lower outer surface of the dispensing rod at positions opposed to each other.

A bead seat recess 142 in which each of the cosmetic cream beads 10 is positioned to be individually dispensed is formed in an upper end surface of the dispensing rod 140.

The intermediate cap 130 fastened to the upper portion of the main body 110 is configured such that a protective cap 150 is fastened to the intermediate cap by screw engagement to so as to protect the intermediate cap against dust and the like.

An annular coupling rim 133 is fastened to the upper coupling portion 114 of the main body 110 by undercut engagement. The discharge recessed portion 132 through which the cosmetic cream beads 10 dispensed by the dispensing rod 140 are exposed is formed on the upper surface of the intermediate cap.

The dispensing rod guide tube 112 of the main body 110 in which the dispensing rod 140 is lifted or lowered has an inner diameter corresponding to an outer diameter of the dispensing rod 140, with a D-cut surface 113 corresponding to a D-cut surface 143 formed on the outer circumferential surface of the dispensing rod 140 such that the dispensing rod 140 can be lifted or lowered without rotation.

The present invention may further include a hermetic sealing ring 160 interposed between an upper end of the dispensing rod lifting cylinder 121 of the rotatable body 120 and the dispensing rod guide tube 112 provided in the center of the bottom surface 111 of the main body 110.

In the bead cream cosmetic storage container 100 having the above-described configuration, the rotatable body 120 is coupled to the lower coupling portion 113 of the main body 110 in which the cosmetic cream beads 10 are accommodated such that the annular locking portion 122a of the edge wall 122 is locked to the annular locking portion (not designated by a specific reference numeral) formed at the coupling portion 113 of the main body 100 at a position therebeyond. Herein, the dispensing rod 140 is assembled to the rotatable body 120 before the rotatable body 120 is coupled to the main body 110.

The assembly of the dispensing rod 140 is performed in such a manner that the guide protrusions 141 formed on the lower portion of the dispensing rod 140 are positioned to be guided along the lifting screw hole 121a of the dispensing rod lifting cylinder 121 provided at the inside center of the rotatable body 120.

Subsequently, the hermetic sealing ring 160 is positioned at the upper edge of the dispensing rod lifting cylinder 121, and then the rotatable body 120 is assembled to the main body 110 as described above.

When the rotatable body 120 is assembled to the main body 110 as described above, the D-cut surface 113a of the dispensing rod 140 assembled to the dispensing rod lifting cylinder 121 is positioned corresponding to the D-cut surface 143 formed at the inner surface of the dispensing rod guide tube 112 of the main body 110.

In the state in which the rotatable body 120 is assembled to the main body 110, the cosmetic cream beads 10 are charged in the main body 110, and then the intermediate cap 130 is assembled to the upper coupling portion 114 by undercut engagement.

The bead cream cosmetic storage container 100 manufactured to store the cosmetic cream beads 10 by assembling the rotatable body 120 and the intermediate cap 130 to the main body 110 as described above will be distributed for sale, with the protective cap 150 covered on the intermediate cap 130.

When a user wants to take out contents of the bead cream cosmetic storage container 100 according to the present invention, the user first removes the protective cap 150 fastened to the intermediate cap 130 by screw engagement.

After removing the protective cap 150, the user holds the rotatable body 120 with one hand while holding the main body 110 with the other hand and rotates the rotatable body in a screw-loosening direction.

Herein, the dispensing rod 140 positioned in the dispensing rod lifting cylinder 121 of the rotatable body 120 is restricted from rotating because the D-cut surface 143 is positioned corresponding to the D-cut surface 112a of the dispensing rod guide tube 112 of the main body 110.

When the rotatable body 120 is rotated in such a state, the guide protrusions 141 of the dispensing rod 140 are guided along the lifting screw hole 121a of the dispensing rod lifting cylinder 121, causing the dispensing rod 140 to be lifted in conjunction with a rotational motion of the rotatable body 120.

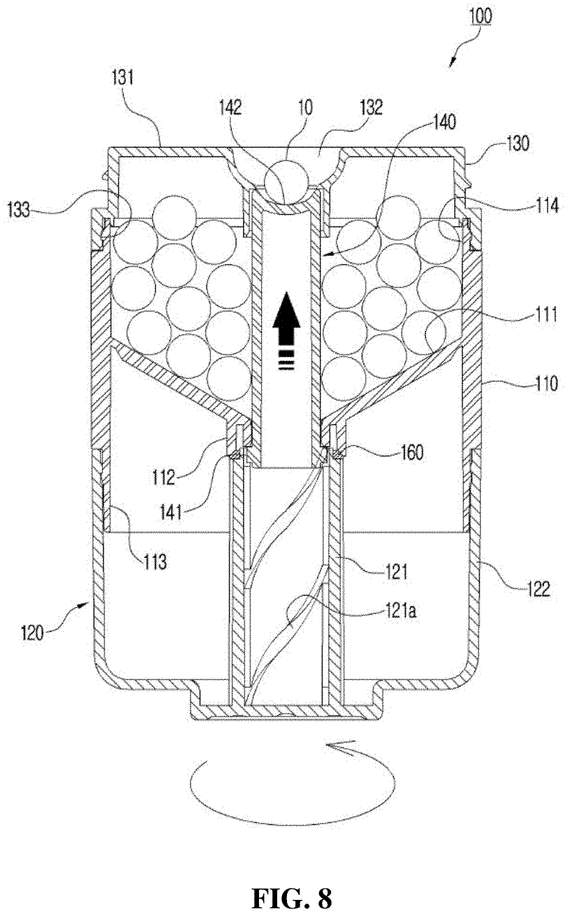

As shown in FIG. 8, the dispensing rod 140 is lifted, with each of the cosmetic cream beads 10 seated in the bead seat recess 142 formed in the upper end surface of the dispensing rod 140, causing each of the cosmetic cream beads to be positioned on the discharge recessed portion 132 formed in the upper surface 131 of the intermediate cap 130.

Each of the cosmetic cream beads 10 positioned as in FIG. 8 is placed on a target area of skin and then rubbed.

Due to the fact that the bottom surface 111 of the main body 110 has a conical shape tapering toward the center thereof, each of the cosmetic cream beads 10 accommodated in the main body 110 is always positioned in the bead seat recess 142 of the dispensing rod 140, regardless of the residual amount of the accommodated cosmetic cream beads 10.

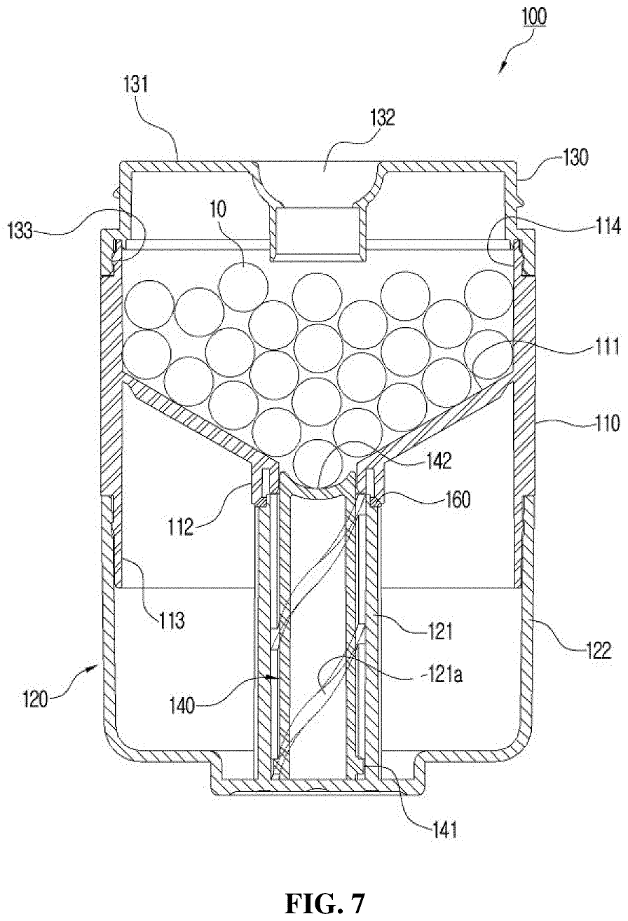

After completing the use of each of the cosmetic cream beads 10 discharged as described above, the user rotates the rotatable body 120 in a direction opposite to the rotation direction of discharging each of the cosmetic cream beads 10, causing the rotatable body to be positioned as shown in FIG. 7 to a position to be able to discharge a next cosmetic cream bead 10.

After that, the protective cap 150 is fastened to the intermediate cap 130 for storage.

1. A bead cream cosmetic storage container that stores and uses, among cosmetic creams used to suppress evaporation of moisture from skin and quickly restore a damaged skin barrier to reduce facial wrinkles and neck wrinkles, cosmetic cream beads (10) each of which includes an outer wax layer (12) and a high content of ceramide (11) contained therein in emulsion form, the bead cream cosmetic storage container comprising:
a main body (110) configured such that a bottom surface (111) forming a space where the cosmetic cream beads (10) are stored has a conical shape to allow the cosmetic cream beads (10) to be gathered to a center of the bottom surface (111), a dispensing rod guide tube (112) in which a dispensing rod (140) pushing up the cosmetic cream beads (10) is positioned to be lifted or lowered is provided in the center of the bottom surface, a rotatable body (120) configured to lift or lower the dispensing rod (140) is assembled to the main body so as to be rotatable via a lower coupling portion (113), and an intermediate cap (130) is coupled to an upper coupling portion (114), the intermediate cap including an upper surface (131) provided with a discharge recessed portion (132) through which the cosmetic cream beads (10) dispensed by the dispensing rod (140) are discharged;
the rotatable body (120) configured such that an upper end thereof is coupled to the dispensing rod guide tube (112) of the main body (110) by concave-convex engagement while an inside center thereof is provided with a dispensing rod lifting cylinder (121) including a lifting screw hole (121a) that is defined by an inner surface thereof and along which a guide protrusion (141) of the dispensing rod (140) is guided, and assembled to the main body by locking engagement of an annular locking portion (122a) formed on an inner surface of an edge wall (122) with an annular locking portion formed on an outer surface of the lower coupling portion (113) of the main body (110);
the dispensing rod (140) configured such that guide protrusions (141) guided along the lifting screw hole (121a) of the dispensing rod lifting cylinder (121) of the rotatable body (120) are formed on a lower outer surface of the dispensing rod at positions opposed to each other, and a bead seat recess (142) in which each of the cosmetic cream beads (10) is positioned to be individually dispensed is formed in an upper end surface of the dispensing rod; and
the intermediate cap (130) configured such that a protective cap (150) is fastened to the intermediate cap by screw engagement so as to protect the intermediate cap against dust and the like, an annular coupling rim (133) is fastened to the upper coupling portion (114) of the main body (110) by undercut engagement, and the discharge recessed portion (132) through which the cosmetic cream beads (10) dispensed by the dispensing rod (140) are exposed is formed on the upper surface of the intermediate cap.

2. The bead cream cosmetic storage container of claim 1, wherein the dispensing rod guide tube (112) of the main body (110) in which the dispensing rod (140) is lifted or lowered has an inner diameter corresponding to an outer diameter of the dispensing rod (140), with a D-cut surface (113) corresponding to a D-cut surface (143) formed on an outer circumferential surface of the dispensing rod (140) such that the dispensing rod (140) can be lifted or lowered without rotation.

3. The bead cream cosmetic storage container of claim 1, further comprising:
a hermetic sealing ring (160) interposed between an upper end of the dispensing rod lifting cylinder (121) of the rotatable body (120) and the dispensing rod guide tube (112) provided in the center of the bottom surface (111) of the main body (110).
